(19) United States
(12) Patent Application Publication
(10) Pub. No.: US 2003/0109917 A1
(43) Pub. Date: Jun. 12, 2003

(54) Stent vascular intervention device and method

(76) Inventors: Stephen Rudin, Williamsville, NY (US); Baruch B. Lieber, Weston, FL (US); Matthew J. Gounis, Miami, FL (US)

Correspondence Address: Michael L. Goldman, NIXON PEABODY LLP, Clinton Square, P.O. Box 31051, Rochester, NY, 14603-1051, US

(21) Appl. No.: 10/199,234

(22) Filed: Jul. 18, 2002

Related U.S. Application Data
(60) Provisional application No. 60/306,200, filed on Jul. 18, 2001.

(51) Int. Cl. A61F 2/06

(52) U.S. Cl. 623/1.15

(57) ABSTRACT

The present invention relates to a stent including a variable porosity, tubular structure having pores defined by structural surfaces. The tubular structure has a low porosity region on a path around the tubular structure, where the low porosity region is less porous than other regions located on the path and fully or partially obstructs passage of fluid. The low porosity region is larger than the structural surfaces between adjacent pores. Also disclosed is a method of altering blood flow within and near an opening of a defective blood vessel involving deploying the above stent of the present invention in a defective blood vessel so that the low porosity region is aligned to and in contact with an opening in the defective blood vessel, thereby altering blood flow within and near the opening of the defective blood vessel.

This application claims the benefit of U.S. Provisional Patent Application Serial No. 60/306,200, filed Jul. 18, 2001, which is hereby incorporated by reference in its entirety.

This work was supported by the National Institute of Health Grant No. 1R01NS38745. The U.S. Government may have certain rights in the invention.

### FIELD OF THE INVENTION

The present invention relates to medical devices, stents in particular, and methods of using high resolution radiographic imaging detectors in endovascular interventions involving stents.

### BACKGROUND OF THE INVENTION

After heart disease and cancer, stroke is the leading cause of death and adult disability in the United States. After stenoses due to plaque or thrombosis, aneurysms and their rupture is the leading cause of stroke. An aneurysm is a bulge in the artery whose cause is not well understood, although most explanations involve a discussion of blood flow and its interaction with the vessel wall. Cerebral aneurysms are most likely to be roughly round berry or saccular shaped rather than fusiform and are most likely to occur near a vessel bifurcation (Hademenos, “Saccular Aneurysm,” The Physics of Cerebrovascular Diseases, Chap. 6.4, p. 183, Springer-Verlag, New York (1998)). What is unique about aneurysms in the cerebrovasculature is that they are often formed in vessels, which have many small but important side branches or perforators. Perforators, typically about 50-250 microns in diameter, are end vessels in that they go directly to a portion of brain tissue with no co-laterals. Hence, they are the only source of blood to these regions. Should perforators be injured or disrupted, impaired brain function or death may occur.

The current treatment for neurovascular aneurysms is either invasive surgical clipping or endovascular embolization (Hademenos, “Treatment for Intracranial Aneurysms,” The Physics of Cerebrovascular Diseases, Chap. 6.8, pp. 215-223, Springer-Verlag, New York (1998); Ringer et al., “Current Techniques for Endovascular Treatment of Intracranial Aneurysms,” in Loftus et al. (eds.) Seminars in Cerebrovascular Disease and Stroke, Vol. 1(1) W. B. Saunders Company (2001)). Because invasive surgical clipping can result in substantial morbidity and mortality, catheter-based interventional procedures are becoming increasingly favored and may be the only treatment possible for some types of lesions deep within the brain. The only presently approved endovascular method is the introduction of short lengths of wire, which have thin hair-like wires sticking out the side giving them a fuzzy appearance. They are also made to bend into specified diameters when they are delivered out of the catheter tip. Thus, it is expected that these “detachable coils” will be wound around the volume of an aneurysm filling the volume of the aneurysm without herniating out into the main blood vessel. If enough of these coils are placed in the aneurysm to disrupt the vortex-like blood flow, it is expected that the blood remaining in the aneurysm adjacent to the coils will thrombose and that a layer of endothelial cells at the neck or entrance to the aneurysm will begin the process of the formation of a new wall to the vessel (Langille, “Blood Flow-Induced Remodeling of the Artery Wall,” in Bevan (eds.) Flow-Dependent Regulation of Vascular Function, Ch. 13, pp. 277-299, Oxford University Press, New York, N.Y. (1995)). The aneurysm, with the coil mass within, is thus sealed off and the main vessel is, in the ideal case, fully recanalized or remodeled to allow normal laminar-like blood flow to resume.

In practice, there are a number of problems with this scenario. The coils may not fully fill the aneurysm volume, since the ones deployed first may interfere with the deployment of the later ones. It may take many coils of different length and diameter to come near to filling the aneurysm volume. A coil may herniate into the main vessel and cause thrombi to form. If these thrombi stay in the main vessel and travel further into the brain, an ischemic stroke may result. Also, one of the coils may inadvertently perforate a weak section of the aneurysm wall resulting in catastrophic hemorrhage. Positioning the final coils may shift the first coils around to undesired positions, either preventing further coiling to completion or possibly causing herniation or perforation. Compaction may commonly occur in time having the effect of incomplete neck filling. The disruption of aneurysmal blood flow may be inadequate and the aneurysm or a new one may regenerate in the same location. Treatment of large and giant aneurysms with coils has been problematic. Additionally, if the aneurysm has a wide neck or is fusiform (bulging on all sides with no clearly defined neck), it may not be possible to introduce coils that will remain within, thus precluding this type of treatment. Finally, there is a growing concern about long-term incomplete endothelialization across the neck resulting from coiling (Bavinzski et al., “Gross and Microscopic Histopathological Findings in Aneurysms of the Human Brain Treated With Guglielmi Detachable Coils,” J. Neurosurg., 91:284-293 (1999); Reul et al., “Long-Term Angiographic and Histopathologic Findings in Experimental Aneurysms of the Carotid Bifurcation Embolized With Platinum and Tungsten Coils,” Am. J. Neuroradiol. 18:35-42 (1997); Kallmes et al., “Histologic Evaluation of Platinum Coil Embolization in an Aneurysm Model in Rabbits,” Radiology, 213:217-222 (1999)).

One advance that is being pursued by Micro Therapeutics, Inc. (Irvine, Calif.) is the use of a liquid polymer material instead of coils. (See http://www.microtherapeutics.com/ for description of the Onyx liquid polymer.) Because the liquid polymer is so viscous, a special high-pressure micro-catheter must be used and placed in the aneurysm, while the orifice of the aneurysm, as well as the main vessel, is blocked by a balloon. The polymer is then introduced into the aneurysm and prevented from escaping into the main vessel by the inflated balloon. The aneurysm is filled in stages every few minutes. Only a few tenths of a milliliter flows into the aneurysm, before the balloon must be deflated to allow blood to resume flowing into the main vessel. Before the next stage, there is a pause while the polymer solidifies after which new liquid polymer is introduced until the aneurysm is finally filled. The balloon does not form a perfect seal to allow displaced blood to leave, but unfortunately at the end of the procedure when the aneurysm is filled, often the polymer flows out over the balloon forming flaps in the main vessel. The potential consequences of this are not known and this procedure is not yet FDA approved. One advantage of the method is that the balloon enables treatment of wide necked aneurysms not possible with coils. The disadvantages aside from the flap formation is the need to repeatedly stop blood flow in the main vessel, the lengthy duration of time needed for the procedure, and the possibility of technical complications such as solidification of the polymer and clogging of the special catheter.

During the attempt to treat wide-necked aneurysms with coils, researchers have tried coils in combination with stents (Szikora et al, “Combined Use of Stents and Coils to Treat Experimental Wide-Necked Carotid Aneurysms: Preliminary Results,” Am. J. Neuroradiol., 15:1091-1102 (1994); Lanzino et al., “Efficacy and Current Limitations of Intravascular Stents for Intracranial Internal Carotid, Vertebral, and Basilar Artery Aneurysms,” J. Neurosurg., 91:538-546 (1999)). Stents are cylindrical scaffolds usually made of stainless steel or nitinol, which are generally used for the treatment of stenoses or vessel narrowing due to atherosclerosis. For application to the endovascular treatment of aneurysms, the stent's function is not one of holding the vessel open but of preventing the coils inserted in an aneurysm from herniating out into the main vessel. The struts of the stent are placed over the orifice of the aneurysm to act as a barrier. Researchers have demonstrated that merely by the deployment of a stent across the ostium of an aneurysm, the characteristic vortex blood flow would be reduced (Lieber et al., “Alteration of Hemodynamics in Aneurysm Models by Stenting: Influence of Stent Porosity,” Annals of Biomed. Eng., 25:460-469 (1997); Aenis et al., “Modeling of Flow in a Straight Stented and Non-Stented Side Wall Aneurysm Model,” J. of Biomech. Eng., 119:206-212 (1997); Livescu et al., “Intra-Aneurysmal Vorticity Reduction Subsequent to Stenting,” Annals of Biomedical Engineering, Vol. 28, Supp. 1:S-61, BMES 2000 Annual Fall Meeting, Seattle, Wash. (2000); Livescu et al., “Influence of Stent Design on Intra-Aneurysmal Flow—A PIV Study,” in Conway (ed.) 2000 Advances in Bioengineering, BED, Vol. 48, ASME Publication:3-4, International Mechanical Engineering Conference & Exposition 2000, Orlando, Fla. (2000); Nichita et al., “Numerical Simulation of Flow in a Stented and Non-Stented Side Wall Aneurysm Model Using the Immersed Boundary Technique,” Annual Meeting of the Society for Mathematical Biology (SMB 2000), Salt Lake City, Utah (2000); Nichita et al., “Numerical Simulation of Flow in a Stented and Non-Stented Cerebral Arterial Segment with a Side Wall Aneurysm Using the Immersed Boundary Technique,” Annals of Biomedical Engineering, Vol. 28, Supp. 1:S-61, BMES 2000 Annual Fall Meeting, Seattle, Wash. (2000)). It was found that the porosity, or open area compared to total outside area of the cylindrical stent, determined how much disruption of the vortex occurred. In one clinical case, where only a stent was deployed with no coils, it was found that the aneurysm actually self-thrombosed (Hopkins et al., “Treating Complex Nervous System Vascular Disorders Through a “Needle Stick”: Origins, Evolution, and Future of Neuroendovascular Therapy,” Neurosurgery, 48:463-475 (2001)). Others have demonstrated similar stent use but in an animal model fusiform aneurysm (Geremia et al., “Occlusion of Experimentally Created Fusiform Aneurysms With Porous Metallic Stents,” Am. J. Neuroradiol., 21(4):739-45 (2000)).

It has become somewhat common practice now to deploy stents in combination with detachable coils. In many such cases, the stent is first deployed and then a microcatheter to deliver the coils is inserted through the openings between the struts of the stent. Nevertheless, many of the potential disadvantages of using coils, such as risk of perforation, long duration of procedure, incomplete filling of the volume, and regrowth of the aneurysm (Hayakawa et al., “Natural History of the Neck Remnant of a Cerebral Aneurysm Treated With the Guglielmi Detachable Coil System,” J. Neurosurg., 93:561-568 (2000)) remain; in addition, there is the new risk to perforator vessels whose orifice may be in close proximity to the aneurysm and hence covered by stent struts. Most recently, there has been a case where adverse effects possibly attributed to blood flow pattern changes occurred. However, detailed flow patterns and consequential wall stress fields, even though generally believed to be crucial to the occurrence, progression, and recurrence after therapy of neurovascular aneurysms (Imbesi et al., “Analysis of Slipstream Flow in a Wide-Necked Basilar Artery Aneurysm: Evaluation of Potential Treatment Regimens, Am. J. Neuroradiol., 22:721-724 (2001); Sorteberg et al., “Effect of Guglielmi Detachable Coils on Intraaneurysmal Flow: Experimental Study in Canines,” Am. J. Neuroradiol., 23:288-294 (2002)) are mostly unexplored.

Because the original primary purpose of stents is to support the wall of diseased vessel rather than modify blood flow, all commercially available stents are uniform and circularly symmetric. Clearly this is not an ideal design for treatment of neurovascular aneurysms which are inherently non-radially symmetric since they are either bulges in the side of a vessel wall or bulges at a vessel bifurcation or fusiform but asymmetric in shape. A stent only needs to be strong enough away from the aneurysm orifice to keep the low porosity section or patch-like region of the new stent in position near or over the aneurysm orifice so as to modify the flow of blood into the aneurysm. This should promote blood stasis and subsequent thrombosis without endangering perforators. A uniformly covered stent would be fatal since it would cover perforators as well as the aneurysm orifice.

The present invention is directed to overcoming these deficiencies in the art.

### SUMMARY OF THE INVENTION

The present invention relates to a stent including a variable porosity, tubular structure having pores defined by structural surfaces. The tubular structure has a low porosity region on a path around the tubular structure, where the low porosity region is less porous than other regions located on the path and fully or partially obstructs passage of fluid. The low porosity region is larger than the structural surfaces between adjacent pores.

Another aspect of the present invention relates to a method of altering blood flow within and near an opening of a defective blood vessel. The method involves deploying the above stent of the present invention in a defective blood vessel so that the low porosity region is aligned to and in contact with an opening in the defective blood vessel, thereby altering blood flow within and near the opening of the defective blood vessel.

The limitations of radiographic visualization and the lack of consideration of the influence of stent deployment on details of blood flow have limited stent design to forms which are uniform and radially symmetric. However, some of the most important potential applications of stents such as in the treatment of aneurysms are inherently non-uniform and non-symmetric in nature. The stents of the present invention are unique in that they are radially asymmetric, have a variable porosity, and are specially designed for flow modification rather than for support of the vessel. Moreover, the ability of new high resolution X-ray image detectors to accurately localize the rotational orientation as well as the longitudinal distance of the stents of the present invention allows for treatment of cerebral aneurysms by modifying aneurysm blood flow characteristics.

The present invention enables less invasive treatment of neurovascular aneurysms with reduced risk of perforation and hemorrhage. It also reduces the likelihood of recurrence compared to existing procedures, and permits treatment of wide-necked, large or giant, and fusiform aneurysms that are presently untreatable. The present invention should have minimal risk to small but crucial perforator vessels unique to the cerebrovasculature. Additionally, the duration of treatment and discomfort to the patient could be vastly reduced, since only one careful stent deployment would constitute the intervention.

### BRIEF DESCRIPTION OF THE DRAWINGS

FIGS. 1A-F depict illustrative designs for the stent of the present invention.

FIGS. 2A-C show how the stent of the present invention can be deployed in a defective blood vessel using a balloon catheter.

### DETAILED DESCRIPTION OF THE INVENTION

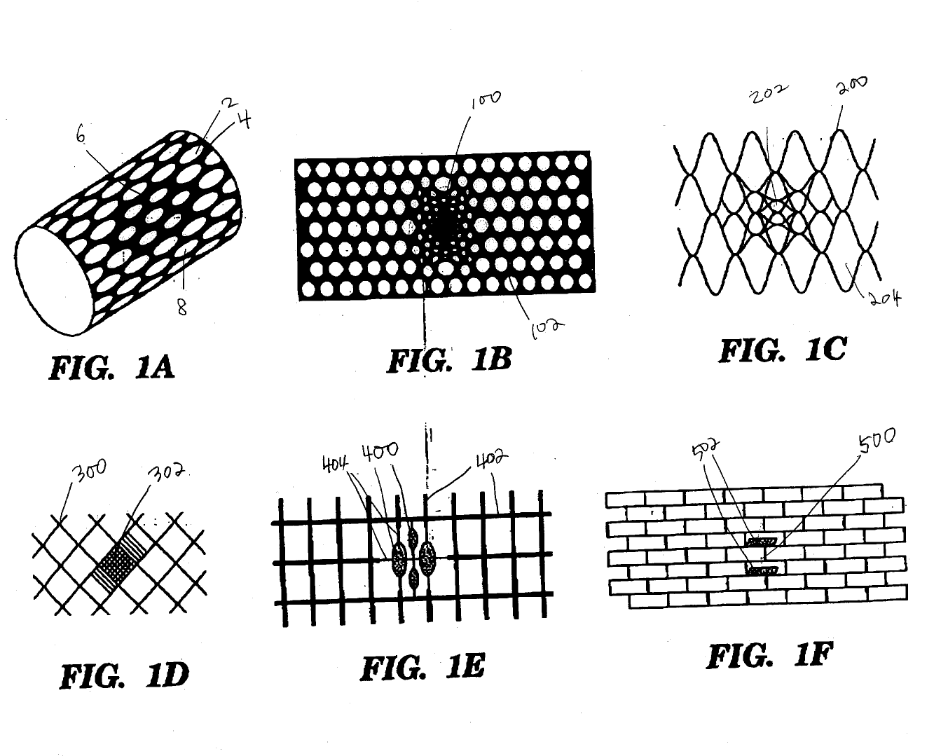

The present invention relates to a stent including a variable porosity, tubular structure having pores defined by structural surfaces. FIGS. 1A-F illustrate different designs for the stent of the present invention.

As shown in FIG. 1A, the tubular structure of the stent of the present invention has low porosity region 6 on a path around the tubular structure, where low porosity region 6 is less porous than other regions 8 located on the path and fully or partially obstructs passage of fluid. Low porosity region 6 is larger than structural surfaces 4 between adjacent pores 2.

In one embodiment of the present invention, the tubular structure of the stent of the present invention has a cylindrical shape and the path is circumferentially around the tubular structure. In another embodiment of the present invention, the tubular structure of the stent of the present invention can be a cylindrical sheet with pores 2 of variable size or shape, as depicted in FIG. 1A. Low porosity region 6 can have a single pore size while all other parts of the tubular structure (e.g. region 8) have another larger pore size, as shown in FIG. 1A.

Alternatively, as depicted in FIG. 1B, low porosity region 100 can have a plurality of pore sizes with the size of the pores increasing as low porosity region 100 transitions to other regions 102 of the stent. The reason to have this type of design for a stent is because of the inaccuracy of positioning the low porosity region of the stent over the entrance opening of the aneurysm. Thus, if the stent were placed inaccurately so that a substantial area of presumably healthy vessel wall is covered and is completely deprived of blood supply, it is possible that there could be deleterious consequences to the vessel. There may not be necrosis, but it is likely that transient apoptosis may take place followed by neointimal hyperplasia, which may give rise to undesired vessel restenosis in the low porosity region of stent. Additionally, if there are perforators near the aneurysm neck, inaccuracies in localizing the stent might cause blockage. By providing the stent with a more gradual change in porosity from the “patch” region to the distal support region, the neointimal reaction might be averted and nearby perforators will not be blocked, even if the accuracy of localization of the stent deployment is not perfect, as long as the aneurysmal blood flow is sufficiently disrupted. In this way, adequate remodeling of the vessel around the stent would still be enabled.

In another embodiment of the present invention, the tubular structure of the stent can be formed from a plurality of strut elements which are thicker, wider, and/or denser in the low porosity region, as shown in FIGS. 1C-E.

FIG. 1C illustrates a stent design which essentially takes a common existing design of connected sine waves or triangle waves, and alters the spacing between strut elements 200 so that a low porosity region 202 is formed. The strut elements can be made of stainless steel. This can be accomplished either by micro-welding or laser-micromachining additional struts to an existing stent, or taking an existing stent with a diameter larger than that of the main channel, then manually bunching some struts together, and under-inflating the stent so that the bunched struts continue to stay together and form low porosity region 202. If the number of struts in higher porosity region 204 is reduced by this bulging process, there are be no adverse consequences because the function of the high porosity region is solely one of supporting the low porosity region rather than keeping the vessel open as would be the case for treatment of stenosis. This design is the easiest to implement almost immediately with existing stent systems.

FIG. 1D illustrates another embodiment of the present invention, where the tubular structure of the stent is made of a mesh material. Here, existing stent 300 is taken and a finer mesh is fastened to form low porosity region 302.

FIG. 1E illustrates another embodiment of the present invention, where the stent is designed to have low porosity region 400 from the start with struts 402 as well as low porosity region 400 having a somewhat uniform strength, which can be carefully micro-machined from uniform cylindrical sheets of material. Low porosity regions 400, which are formed by thicker parts 400 of struts 402, are connected by thinner parts 404 of struts 402 so that the overall strut strength is uniform. In theory, this design may be the most optimal of the designs, but it will be the most difficult to machine. In addition, a stent with this design may be difficult to crimp onto balloons and hence may take the longest development time.

In yet another embodiment of the present invention, low porosity region 500 of the stent is formed by flap-like structures 502 in the pores. FIG. 1F depicts a stent having active flow diverters 502, which could be deployed or changed in the field to obstruct fluid flow.

The stent of the present invention can be balloon expandable so that it can be deployed using a balloon catheter. Alternatively, the stent of the present invention can be self-expandable where the stent is made of a shape memory material and can be deployed by self-expansion. Shape memory materials can be annealed into a first shape, heated, thereby setting the material structure, cooled, and deformed into a second shape. The material returns to the first, remembered shape at a phase transition temperature specific to the material composition. Shape memory materials include, for example, nickel-titanium alloy, which is available under the name of nitinol.

Another aspect of the present invention relates to a method of altering blood flow within and near an opening of a defective blood vessel. The method involves deploying the stent of the present invention in a defective blood vessel so that the low porosity region is aligned to and in contact with an opening in the defective blood vessel, thereby altering blood flow within and near the opening of the defective blood vessel.

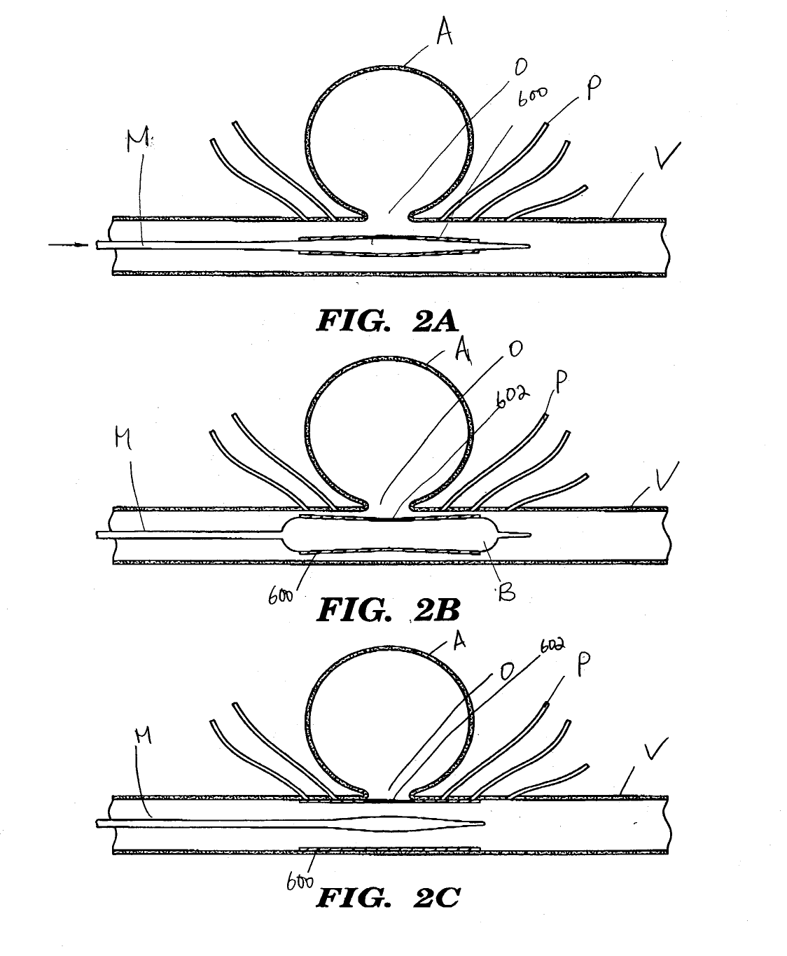

Balloon expansion and self-expansion are the most common methods of deploying stents. Balloon expansion, which is more compact, is particularly useful for small cerebral vessels. FIGS. 2A-C depict the steps necessary for deploying the stent of the present invention by the balloon expansion method. In FIG. 2A, stent 600 is shown with balloon microcatheter M inserted inside for deployment in a defective blood vessel V near opening O of aneurysm A. FIG. 2B shows partially deployed stent 600 where stent 600 is being expanded by balloon part B of balloon microcatheter M after low porosity region 602 of stent 600 is aligned to and in contact with opening O of aneurysm A. As shown in FIG. 2C, after stent 600 is fully deployed in the desired location, balloon part B of balloon microcatheter M is collapsed to release stent 600 and ballon microcatheter M exits blood vessel V.

With respect to treating cerebrovascular aneurysms by inserting flow modifying devices such as stents by minimally invasive, catheter-based methods, it is important that the small but crucial side-branch perforating vessels unique to the cerebrovasculature be minimally damaged or blocked. As shown in FIG. 2C, stent 600 of the present invention is deployed in defective blood vessel V so that low porosity region 602 is placed across opening O of aneurysm A to disrupt the aneurysmal blood flow, while the other higher porosity regions of the stent do not block perforating vessels P.

The stent of the present invention can also be deployed by self-expansion of the stent. Thus, a stent made of a shape memory material can be used, where the stent is compressed to fit within a microcatheter, delivered to the aneurysm, and pushed from the microcatheter end. Subsequently, the stent regains its uncompressed shape, where the low porosity region of the stent is aligned to and in contact with the opening in the defective blood vessel so as to modify blood flow into the aneurysm.

Part of the difficulty in present applications of stents to the cerebral vasculature is the difficulty in navigating a somewhat rigid undeployed stent through tortuous vasculature to the lesion. Part of the reason for rigidity in stents is the requirement for treatment of stenoses that the stent maintain sufficient hoop strength to keep the vessel in question open. For application to aneurysms, however, this requirement for rigidity can be relaxed because the sole function of the stent is to keep the low porosity region in the proper place like a “patch,” as in the present invention.

In order to correctly deploy the stent of the present invention at the opening of an aneurysm, one would need a way to visualize the asymmetric part of the stent (i.e. the low porosity region). Thus, the new stent will have to be positioned accurately both in the direction of the catheter axis and also in rotational angle, so as to align the low porosity region of the stent with the aneurysm orifice. Therefore, another embodiment of the present invention relates to using high resolution radiographic imaging to guide the deployment of the stent of the present invention. U.S. Pat. No. 6,285,739 to Rudin et al., which is hereby incorporated by reference in its entirety, discloses high resolution micro-angiographic detectors for viewing a limited region of interest near the interventional site, usually at the catheter tip, which can be used to provide the necessary guidance for accurate rotational orientation of the stent in the blood vessel.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A stent comprising:
a variable porosity, tubular structure having pores defined by structural surfaces, said tubular structure having a low porosity region on a path around the tubular structure, wherein the low porosity region is less porous than other regions located on the path and fully or partially obstructs passage of fluid, the low porosity region being larger than the structural surfaces between adjacent pores.

2. The stent of claim 1, wherein said tubular structure comprises a cylindrical sheet with pores of variable size or shape.

3. The stent of claim 2, wherein said tubular structure is made of a mesh material.

4. The stent of claim 1, wherein the low porosity region has a single pore size while all other parts of the tubular structure have another larger pore size.

5. The stent of claim 1, wherein the low porosity region has a plurality of pore sizes with the size of the pores increasing as the low porosity region transitions to other regions of the stent.

6. The stent of claim 1, wherein said tubular structure is formed from a plurality of strut elements which are thicker, wider, and/or denser in the low porosity region.

7. The stent of claim 6, wherein the strut elements are made of stainless steel.

8. The stent of claim 1, wherein the stent is balloon expandable.

9. The stent of claim 1, wherein the low porosity region is formed by flap-like structures in the pores.

10. The stent of claim 1, wherein the stent is made of a shape memory material so that the stent is expandable.

11. The stent of claim 10, wherein the shape memory material is nitinol.

12. The stent of claim 1, wherein the tubular structure has a cylindrical shape and the path is circumferentially around the tubular structure.

13. A method of altering blood flow within and near an opening of a defective blood vessel comprising:
deploying the stent of claim 1 in a defective blood vessel so that the low porosity region is aligned to and in contact with an opening in the defective blood vessel, thereby altering blood flow within and near the opening of the defective blood vessel.

14. The method of claim 13, wherein said deploying is performed using a balloon catheter.

15. The method of claim 13, wherein said deploying is performed by self-expansion of the stent.

16. The method of claim 13, wherein said deploying is guided by high resolution radiographic imaging.

17. The method of claim 13, wherein the tubular structure of said stent comprises a cylindrical sheet with pores of variable size or shape.

18. The method of claim 17, wherein said tubular structure is made of a mesh material.

19. The method of claim 13, wherein the low porosity region has a single pore size while all other parts of the tubular structure have another larger pore size.

20. The method of claim 13, wherein the low porosity region has a plurality of pore sizes with the size of the pores increasing as the low porosity region transitions to other regions of the stent.

21. The method of claim 13, wherein the tubular structure of said stent is formed from a plurality of strut elements which are thicker, wider, and/or denser in the low porosity region.

22. The method of claim 21, wherein the strut elements are made of stainless steel.

23. The method of claim 13, wherein the low porosity region is formed by flap-like structures in the pores.

24. The method of claim 13, wherein the tubular structure has a cylindrical shape and the path is circumferentially around the tubular structure.
